SEQUENCE LISTING (8 sequences)

SEQ ID NO: 1 - protein, 26 aa, Homo sapiens
SIPENNIMRTIIEFLSFLHLKEAGAL

SEQ ID NO: 2 - protein, 41 aa, Homo sapiens
ELRPEDDMKPGSFDRSIPENNIMRTIIEFLSFLHLKEAGAL

SEQ ID NO: 3 - protein, 59 aa, Homo sapiens
ELRPEDDMKPGSFDRSIPENNIMRTIIEFLSFLHLKEAGALDRLLDLPAAASSEDIERS

SEQ ID NO: 4 - protein, 59 aa, Macaca mulatta
ELQPQDDVKPGSFDRSMPENNIMRTIIEFLSFLHLKEAGAFDRLPDLPAGASSEDMERS

SEQ ID NO: 5 - protein, 59 aa, Bos taurus
ELEPEDEARPGSFDRPLAENNVVRTIIEFLTFLHLKDAGALERLPSLPTAESAEDAERS

SEQ ID NO: 6 - protein, 54 aa, Mus musculus
EERRPGSVDVPLPESNIVRTIMEFLSFLHLKEAGALDSLPGIPLATSSEDLEKS

SEQ ID NO: 7 - protein, 59 aa, Sus scrofa
ELEPEDEARPGGFDRLQSEDKAIRTIMEFLAFLHLKEAGALGRLPGLPSAASSEDAGQS

SEQ ID NO: 8 - protein, 60 aa, Canis familiaris
ELPPEDEGRSGGFAGPLSLSENAAVRMLIEFLTFLRLKEAGALPDLPDLPSAVSAEDMEQ

(19) United States
(12) Patent Application Publication
(10) Pub. No.: US 2009/0317395 A1
(43) Pub. Date: Dec. 24, 2009

(54) Antimicrobial Peptide Derived from Galanin Message Associated Peptide (GMAP)

(76) Inventors: Barbara Kofler, Salzburg (AT); Isabella Rauch, Mondsee (AT)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/306,989

(22) PCT Filed: Jun. 27, 2007
(86) PCT No.: PCT/EP2007/005693

§ 371 Date: May 21, 2009

(30) Foreign Application Priority Data
Jul. 11, 2006 (EP) 06014350.0

(51) Int. Cl. A61K 38/16; C07K 7/06; C07K 7/08; C07K 14/00; C07K 16/00; C07H 21/04; C12N 15/63; C12N 5/10; G01N 33/53; A61K 38/08; A61K 38/10; A61K 31/7088; A61K 39/395; A61P 31/00

(52) U.S. Cl. 424/139.1; 530/328; 530/327; 530/326; 530/325; 530/324; 530/350; 530/323; 530/387.9; 536/23.5; 435/320.1; 435/325; 435/7.1; 514/2; 514/16; 514/15; 514/14; 514/13; 514/12; 514/44 R

(57) ABSTRACT

The present invention relates to a new antimicrobial peptide, comprising the amino acid sequence of human galanin message associated peptide (GMAP) or a homolog, ortholog, chemically modified variant or antimicrobially active variant thereof, and respective uses thereof, particular in the topical treatment of antimicrobial diseases.

The present invention relates to a new antimicrobial peptide, comprising the amino acid sequence of human galanin message associated peptide (GMAP) or a homolog, ortholog, chemically modified variant or antimicrobially active variant thereof, and respective uses thereof, in particular in the topical treatment of antimicrobial diseases.

For the purposes of the present invention, all references as cited herein are incorporated by reference in their entireties.

### BACKGROUND OF THE INVENTION

Several epithelial surfaces are continuously exposed to potential pathogens but rarely become infected, because they are protected by specific secretions. Antimicrobial peptides (AMPs) participate in this innate immune response by providing a rapid first line defense against infection.

The ability of a multicellular organism to defend itself against invasion by pathogens (bacteria, fungi, viruses, etc.) depends on its ability to mount immune responses. All metazoans have inborn defense mechanisms that constitute innate immunity. Vertebrates have not only innate immunity but also are able to mount defense mechanisms that constitute adaptive immunity. The vast majority of microorganisms are destroyed within minutes or hours by innate defenses. The acquired specific immune response comes into play only if these innate defenses are breached.

The innate immunity comprises:

### a) Anatomic Barriers

Skin (physical barrier, low pH due to lactic and fatty acids)
Epidermis—thin outer layer containing tightly packed epidermal cells and keratin (water-proofing) completely renewed every 15-30 days.
Dermis—thicker inner layer contains sebaceous glands associated with hair follicles—produce sebum which consists of lactic and fatty acids maintaining a pH 3-5.
Mucous membranes (ciliated epithelial cells; saliva, tears and mucous secretions)—GI, urogenital, respiratory tracts—collectively represents a huge surface area.

### b) Physiologic Barriers

Temperature—normal body temperature inhibits growth of most microorganisms.
Elevated body temperature (fever) can have a direct effect on pathogenic microorganisms.
pH—low pH of stomach, skin, & vagina (inhibits microbial growth).
Oxygen tension.

### c) Chemical Barriers (Only a Few Examples are Given):

Fatty acids, lactic acid.
Pepsin (digestive enzyme which hydrolyzes proteins).
Lysozyme—hydrolytic enzyme found in mucous secretions—able to cleave the peptidoglycan layer of the bacterial cell wall.
Interferon's—group of proteins produced by cells following viral infection. Secreted by the cells, and then binds to nearby cells and induces mechanisms which inhibit viral replication.
Complement—a group of serum proteins that circulate in an inactive proenzyme state. These proteins can be activated by a variety of specific and non-specific immunologic mechanisms that convert the inactive proenzymes into active enzymes. The activated complement components participate in a controlled enzymatic cascade that results in membrane-damaging reactions, which destroy pathogenic organisms by formation of a membrane attack complex (MAC).
Anti-microbial substances which directly destroy microorganisms: cryptdins and \acute{α}-defensins (produced in base of crypts of small intestine—damage cell membranes) β-defensins (produced within skin, respiratory tract—also damages cell membranes) surfactant proteins A & D (present in lungs—function as opsonins which enhance the efficiency of phagocytosis).

Once a pathogen penetrates the skin or mucosal epithelium, it usually establishes a local infection. Tissue damage and pathogen antigens (Antigens) signal tissue macrophages to secrete chemotactic cytokines called chemokines to attract additional phagocytes and allow more fluid and cells to enter the tissues at the infection site. Neutrophils and macrophages engulf pathogens and destroy them. The alternative complement cascade is activated on pathogen surfaces to promote phagocytosis and pathogen lysis. Anaphylatoxins C3a and C5a attract more leukocytes and increase capillary leakiness at the infection site, allowing phagocytes and complement to enter the tissues. Inflammation is the influx of fluid and cells that results in redness, swelling, heat, and pain (rubor, tumor, calor, dolor) at the infection site. If the innate immune response does not rapidly eliminate pathogen, adaptive immune responses are stimulated.

Neuropeptides are expressed by neuronal and non-neuronal tissues in various organs including the skin, which constitutes the first barrier against external stress. Neuropeptides like corticotrophin releasing hormone (CRH) and pro-opiomelanocortin (POMC)-derived peptides are considered to be involved in preservation of the cutaneous and, in consequence, body homeostasis. These neuropeptides exert potent growth- and immunomodulatory effects as well as antimicrobial activity.

All known neuropeptides possess multiple functionalities. They are involved in the transmission of signals not only between nerve cells, but also the nervous and the immune system (Hokfelt T, Bartfai T and Bloom F: Neuropeptides: opportunities for drug discovery. Lancet Neurol. 2:463-472., 2003) where they appear to be critical mediators of physiological processes. In the skin, neuropeptides are released in response to nociceptive stimulation by pain, temperature, mechanical and chemical irritants to mediate skin responses to combat infection and injury and promote wound healing (Luger T A and Lotti T: Neuropeptides: role in inflammatory skin diseases. J Eur Acad Dennatol Venereal. 10:207-211, 1998). In addition, neuropeptides were also detected in non-neuronal cells of the skin including fibroblasts, keratinocytes and immune cells (Lotti T, Hautmann G and Panconesi E: Neuropeptides in skin. J Am Acad Dermatol. 33:482-496, 1995).

Antimicrobial Peptides (AMPs) participate in the innate immune response by providing a rapid first-line of defense against pathogens. Mucosal secretions, phagocytes and other components of the innate host defense initiate the response to microbial penetration before adaptive immunity starts to work. As effectors of innate immunity, AMPs directly kill a broad spectrum of bacteria, fungi and certain viruses by attacking their cell wall (Bardan A, Nizet V and Gallo R L: Antimicrobial peptides and the skin. Expert Opin Biol Ther 4543-549, 2004; Ulvatne H: Antimicrobial peptides: potential use in skin infections. Am J Clin Dermatol 4:591-595, 2003). This is facilitated by the only property these peptides share among themselves, the affinity for negatively charged phospholipids. In addition, these peptides modify the local inflammatory response and activate mechanisms of adaptive immunity. In the skin, two major groups, cathelicidins and defensins have been studied most extensively (Bardan A, Nizet V and Gallo R L: Antimicrobial peptides and the skin. Expert Opin Biol Ther 4543-549, 2004). The latter is also produced in the central nervous system and inducible by cytokines and lipopolysaccarides (LPS) (Hao H N, Zhao J, Lotoczky G, Grever W E and Lyman W D: Induction of human beta-defensin-2 expression in human astrocytes by lipopolysaccharide and cytokines. J Neurochem. 77:1027-1035., 2001).

The fact that neuropeptides, like AMPs are in general amphipathic molecules may explain the recent findings of several neuropeptides with antimicrobial activity:

1) Adrenomedullin is expressed in various anatomical sites including the skin and shows high similarity to cecropin, an insect-antibiotic peptide. It regulates proliferation, natriuresis and secretion of other hormones and inhibits growth of bacteria in picomolar concentrations (Allaker R P, Zihni C and Kapas S: An investigation into the antimicrobial effects of adrenomedullin on members of the skin, oral, respiratory tract and gut microflora. FEMS Immunol Med Microbiol 23:289-293, 1999). Pro-inflammatory cytokines like IL-1 and lipopolysaccharide (LPS) or exposure to microbes induce expression of the gene coding for the precursor protein of adrenomedullin (Zaks-Zilberman M, Salkowski C A, Elsasser T, Cuttitta F and Vogel S N: Induction of adrenomedullin mRNA and protein by lipopolysaccbaride and paclitaxel (Taxol) in murine macrophages. Infect Immun. 66:4669-4675., 1998). This precursor also contains a second peptide called proadrenomedullin amino-terminal 20 peptide (PAMP), which is active against gram-negative bacteria.
2) Alpha-melanocyte stimulating hormone (α-MSH), another neuropeptide expressed in the skin (Schauer E, Trautinger F, Kock A, et al: Proopiomelanocortin-derived peptides are synthesized and released by human keratinocytes. J Clin Invest. 93:2258-2262., 1994), was already known to have anti-inflammatory and antipyretic effects when its antimicrobial activity was discovered. A carboxyterminal fragment of α-MSH displayed an even higher activity than the original peptide (Cutuli M, Cristiani S, Lipton J M and Catania A: Antimicrobial effects of alpha-MSH peptides. J Leukoc Biol 67:233-239, 2000).
3) Neuropeptide Y (NPY) is an amidated peptide of 36 amino acids and is known to regulate physiological functions, such as food intake, learning behaviour, vasoconstriction and neurotransmitter release (Lin S, Boey D and Herzog H: NPY and Y receptors: lessons from transgenic, and knockout models. Neuropeptides 38: 189-200, 2004). In addition to these functions, NPY has been recently reported to have antifungal activity (Shimizu M, Shigeri Y, Tatsu Y, Yoshikawa S and Yumoto N: Enhancement of antimicrobial activity of neuropeptide Y by N-terminal truncation. Antimicrob Agents Chemother 42:2745-2746, 1998). NPY is suggested to belong to a group of antimicrobial peptides such as dermaseptins, cecropins or magainins, which are helical and devoid of cysteine.
4) Other neuropeptides found to have antimicrobial activity are substance P, corticostatin RK-1, neurotensin, bradykinin and enkelytin (Bateman A, MacLeod R J, Lembessis P, Hu J, Esch F and Solomon S: The isolation and characterization of a novel corticostatin/defensin-like peptide from the kidney. J Biol Chem. 271:10654-10659., 1996; Goumon Y, Lugardon K, Kieffer B, Lefevre J F, Van Dorsselaer A, Aunis D and Metz-Boutigue M H: Characterization of antibacterial COOH-terminal proenkephalin-A-derived peptides (PEAP) in infectious fluids. Importance of enkelytin, the antibacterial PEAP209-237 secreted by stimulated chromatin cells. J Biol Chem. 273:29847-29856, 1998; Kowalska K, Carr D B and Lipkowski A W: Direct antimicrobial properties of substance P. Life Sci 71:747-750, 2002).

Microbial resistance to conventional antibiotics has become commonplace, resulting in escalating numbers of infections with multi-resistant bacteria. Especially in hospitals nosocosmial infections with Staphylococcus aureus, E. coli and Pseudomonas aeruginosa leave only few therapeutic options. Recent studies with AMPs as mediators of the innate host defense have shown, that unlike pharmacologic antibiotics, these native host defense molecules have maintained broad-spectrum antimicrobial activity. Since the AMP induced changes of the phospholipid organization their membrane are difficult for microbes, development of resistance occurs at a very low level. Since these peptides are derived from the human body, there is less risk of unexpected side effects. Molecules that mimic the action of AMPs and molecules that block their degradation could have significant therapeutical potential (Gallo R L, Murakami M, Ohtake T and Zaiou M: Biology and clinical relevance of naturally occurring antimicrobial peptides. J Allergy Clin Immunol 110:823-831, 2002; Ulvatne H: Antimicrobial peptides: potential use in skin infections. Am J Clin Dermatol 4:591-595, 2003). Especially in conditions of an undeveloped or compromised immune system, as seen in neonatology or under immune-suppression as seen in oncology or HIV infection, alternative treatments of microbial infection are desirable. The synthetic homolog of permeability increasing protein has been used successfully to treat children with severe meningococcal sepsis (Giroir B P: New therapies for severe meningococcal disease. Lancet 351 526-528, 1998; Giroir B P, Quint P A, Barton P, et al: Preliminary evaluation of recombinant amino-terminal fragment of human bactericidal/permeability-increasing protein in children with severe meningococcal sepsis. Lancet 350:1439-1443, 1997; Levin M, Quint P A, Goldstein B, et al: Recombinant bactericidal/permeability-increasing protein (rBPI21) as adjunctive treatment for children with severe meningococcal sepsis: a randomised trial. rBP121 Meningococcal Sepsis Study Group. Lancet 356:961-967, 2000).

WO 00/59527 describes an octapeptide based on the sequence of α-MSH-fragments having antifungal activity and affecting C. albicans growth for the treatment of candidiasis (Zengen Inc.).

EP 0 587 571 B1 describes a peptide having the amino acid sequence of human galanin. The amino acid sequence of a particularly claimed peptide is: GWTLNSAGYLLGPHAVGNHRSFSDKNGLTS. EP 0587 571 B1 further describes DNA clones encoding the peptide and to therapeutic uses of the peptide in connection with insulin-production and appetite.

There is a constant need in the art to identify and provide antimicrobial agents that are useful for improved antimicrobial treatments. It is therefore an object of the present invention, to provide such a new antimicrobial agent. Other related objects will become apparent to the person of skill from reading the following description and examples.

According to the present invention, the object of the present invention is solved by providing an antimicrobial peptide, comprising the amino acid sequence of human galanin message associated peptide (GMAP) amino acids 16-41 (SIPENNIMRTIIEFLSFLHLKEAGAL, SEQ ID No. 1), or a homolog, ortholog, chemically modified variant or antimicrobially active variant thereof, wherein the peptide is not complete GMAP (i.e. one of the full-length sequences of GMAP proteins as described in the literature). Preferably, said peptide is isolated.

Preferred is an antimicrobial peptide according to the invention, comprising the amino acid sequence of human galanin message associated peptide (GMAP) amino acids 1-41 (ELRPEDDMKPGSFDRSIPENNIMRTIIEFLSFLHLKEAGAL, SEQ ID No. 2), or a homolog, ortholog, allelic variant, chemically modified variant or antimicrobially active variant thereof, wherein the peptide is not complete GMAP.

The term “ortholog” (or “species homolog”) denotes a polypeptide or protein obtained from one species that has homology to an analogous polypeptide or protein from a different species. The term “paralog” denotes a polypeptide or protein obtained from a given species that has homology to a distinct polypeptide or protein from that same species.

The term “allelic variant” denotes any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

In the context of the present invention, a “chemically modified variant” shall mean a peptide, wherein the amino acids of the sequence have been modified to include additional chemical moieties. Usually, this modification is performed in vitro and introduces chemical groups, such as dyes, linkers, spacers, enzymes, and the like, that allow for improved handling of the peptides (such as coupling to a solid phase or dye-based reaction) or the more effective generation of antibodies (such as with poly-lysine-moieties). The chemically modified variant of the peptide according to the invention will have substantially the same conformation and/or function (i.e. confers antimicrobial activity), as the non-modified peptide according to the invention.

In the context of the present invention, an “antimicrobially active variant” shall mean a peptide that can be a homolog, ortholog, allelic variant, chemically modified variant as defined above or otherwise modified (for example mutated and/or truncated) peptide of the present invention, which, nevertheless, maintains its antimicrobial activity if tested in an antimicrobial assay, such as, for example, described herein below.

“Antimicrobial” shall mean an activity against microbes, such as prokaryotic or eukaryotic microbes, preferably fungi, such as yeasts, or bacteria, such as gram-negative E. coli, gram-positive Staphylococcus aureus and Corynebacterium sp. Preferably, the peptide according to the invention is active against the yeast Candida albicans.

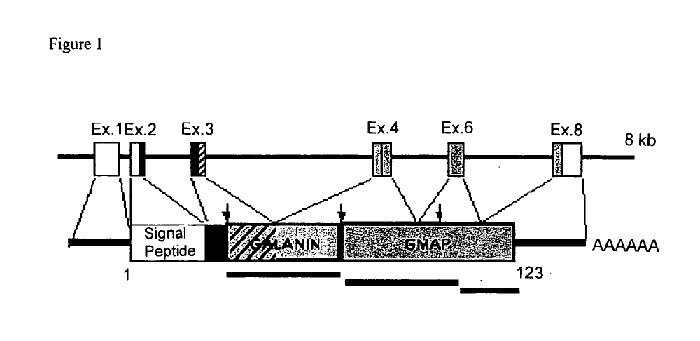

Galanin (GAL), a 29-amino acid peptide, was initially isolated from porcine intestine in 1983 (Tatemoto K, Rokaeus A, Jomvall H, McDonald T J and Mutt V: Galanin—a novel biologically active peptide from porcine intestine. FEBS Lett 161:124-128, 1983). GAL is processed of a 123-amino-acid precursor molecule pre-pro galanin (ppGAL), which contains a signal peptide, mature galanin peptide and a carboxy-terminal 59-amino-acid galanin message associated peptide (GMAP; FIG. 1). For GMAP, no major physiological functions and receptors have been identified. The full length sequence of human galanin is described in the database under Accession numbers CAA01907 (25 Sep. 1995) and P22466 (13 Jun. 2006; Galanin precursor [Contains: Galanin; Galanin message-associated peptide (GMAP)].

Giorgianni, F., Beranova-Giorgianni, S, and Desiderio, D. M. in “Identification and characterization of phosphorylated proteins in the human pituitary” (Proteomics 4 (3), 587-598 (2004)) describe a phosphorylation of galanin at Ser-117 in a pituitary sample.

Further preferred is an antimicrobial peptide according to the invention, wherein said peptide is an antimicrobial peptide according to the invention that is derived from an amino acid sequence according to SEQ ID No. 3 to SEQ ID No. 8 or a chemically modified variant thereof, wherein the peptide is not complete GMAP. Preferred peptides are peptides with amino acids 1 to 41 or amino acids 16 to 41 of the SEQ ID No. 3 to SEQ ID No. 8. The following table shows preferred antimicrobial peptides of the present invention, as well as their origins:

| | | |
|---|---|---|
| GMAP Protein | Sequence | SEQ ID No. |

| | | |
|---|---|---|
| Homo sapiens | ELRPEDDMKPGSFDRSIPENNIMRTIIEFLSFLHLKEAGALDRLLD | SEQ ID No. 3 |
| | LPAAASSEDIERS | |
| Macaca | ELQPQDDVKPGSFDRSMPENNIMRTIIEFLSFLHLKEAGAFDRLPD | SEQ ID No. 4 |
| mulatta | LPAGASSEDMERS | |
| Bos taurus | ELEPEDEARPGSFDRPLAENNVVRTIIEFLTFLHLKDAGALERLPS | SEQ ID No. 5 |
| | LPTAESAEDAERS | |
| Mus musculus | EERRPGSVDVPLPESNIVRTIMEFLSFLHLKEAGALDSLPGIPLAT | SEQ ID No. 6 |
| | SSEDLEKS | |
| Sus scrofa | ELEPEDEARPGGFDRLQSEDKAIRTIMEFLAFLHLKEAGALGRLPG | SEQ ID No. 7 |
| | LPSAASSEDAGQS | |
| Canis | ELPPEDEGRSGGFAGPLSLSENAAVRMLIEFLTFLRLKEAGALPDL | SEQ ID No. 8 |
| familiaris | PDLPSAVSAEDMEQ | |

“Consisting essentially of” shall mean that a peptide according to the present invention, in addition to the sequence according to any of SEQ ID No. 1 to SEQ ID No. 8 or a chemically modified variant thereof, contains additional N- and/or C-terminally located stretches of amino acids that are not necessarily forming part of the peptide that functions as antimicrobial peptide core sequence as defined herein.

Recently, the inventors have gained evidence that the neuropeptide galanin (GAL) is one of the few neuropeptides expressed in non-neuronal cells of the human skin including keratinocytes. The expression of the galanin precursor peptide mRNA (ppGAL) in human epidermis, hair follicles and sweat glands as well as the up-regulation of ppGAL gene expression upon cytokine treatment of human keratinocytes indicated a role in defense mechanisms to external injury and/or invasion. Indeed, a C-terminal part of the ppGAL precursor peptide GMAP possesses anti-fungal activity. The analysis of proteolytic processing of ppGAL in cultured human keratinocytes and the use of synthetic GMAP fragments in antimicrobial assays revealed the core sequence of GMAP responsible for antimicrobial activity.

The peptides according to the invention can have an overall length of between 8 and 100, preferably between 15 and 50, and most preferred between 20 and 30 amino acids. Furthermore, at least one peptide according to any of SEQ ID No. 1 to SEQ ID No. 8 can include non-peptide bonds. Furthermore, the respective nucleic acids can encode for between 8 and 100, preferably between 15 and 50, and most preferred between 20 and 30 amino acids. Most preferred are antimicrobial peptides according to SEQ ID No. 1 or 2 or derived from SEQ ID No. 3 to 8 or a chemically modified variant thereof.

In a further preferred embodiment thereof, the invention provides an antimicrobial peptide, wherein said peptide includes non-peptide bonds.

By “peptide” the inventors include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immunol. 159, 3230-3237, incorporated herein by reference. This approach involves making pseudopeptides containing changes involving the backbone, and not the orientation of side chains. Retro-inverse peptides, which contain NH—CO bonds instead of CO—NH peptide bonds, are much more resistant to proteolysis.

Peptides (at least those containing peptide linkages between amino acid residues) may be synthesized by the Fmoc-polyamide mode of solid-phase peptide synthesis as disclosed by Lu et al (1981) J. Org. Chem. 46, 3433-3436, and references therein. Temporary N-amino group protection is afforded by the 9-fluorenylmethyloxycarbonyl (Fmoc) group. Repetitive cleavage of this highly base-labile protecting group is achieved by using 20% piperidine in N,N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydrol group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalizing agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N,N-dicyclohexyl-carbodiimide/1hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used are ethanedithiol, phenol, anisole and water, the exact choice depending on the constituent amino acids of the peptide being synthesized.

Trifluoroacetic acid is removed by evaporation in vacuo, with subsequent trituration with diethyl ether affording the crude peptide. Any scavengers present are removed by a simple extraction procedure which on lyophilization of the aqueous phase affords the crude peptide free of scavengers. Reagents for peptide synthesis are generally available from Calbiochem-Novabiochem (UK) Ltd, Nottingham NG7 2QJ, UK.

Purification may be effected by any one, or a combination of, techniques such as size exclusion chromatography, ion-exchange chromatography and (usually) reverse-phase high performance liquid chromatography.

Analysis of peptides may be carried out using thin layer chromatography, reverse-phase high performance liquid chromatography, amino-acid analysis after acid hydrolysis and by fast atom bombardment (FAB) mass spectrometric analysis, as well as MALDI and ESI-Q-TOF mass spectrometric analysis.

The term “isolated” polypeptide or peptide as used herein refers to a polypeptide or a peptide which either has no naturally-occurring counterpart or has been separated or purified from components which naturally accompany it, e.g., in tissues such as cortex, olfactory bulb, brainstem, cerebellum, hypothalamus, pituitary gland, adrenal gland, and/or thymus, or body fluids such as blood, serum, or urine. Typically, the polypeptide or peptide is considered “isolated” when it is at least 70%, by dry weight, free from the proteins and other naturally-occurring organic molecules with which it is naturally associated. Preferably, a preparation of a polypeptide (or peptide) of the invention is at least 80%, more preferably at least 90%, and most preferably at least 99%, by dry weight, the polypeptide (or the peptide), respectively, of the invention. Thus, for example, a preparation of peptide x is at least 80%, more preferably at least 90%, and most preferably at least 99%, by dry weight, peptide x. Since a peptide that is chemically synthesized is, by its nature, separated from the components that naturally accompany it, the synthetic peptide is “isolated.”

In a further preferred embodiment thereof, the invention provides an antimicrobial peptide, wherein said peptide is a fusion protein. Fusion proteins and methods for their construction are well known in the state of the art and can include genetic fusion of the peptide with an enzyme group (e.g. producing a color signal), a tag for purification (e.g. a His-tag), a chelating peptide, and the like.

In another aspect of the present invention, there are also provided antisera and an antibody or fragment thereof that is immunologically reactive with the antimicrobial peptide of the present invention which also can be utilized in methods of treatment which involve competitive inhibition of the attachment of the antimicrobial peptide to a microorganism. In particular, specific polyclonal antiserum or an antibody or fragment thereof against the antimicrobial peptide could be generated that reacts with the antimicrobial peptide in, for example, Western immunoblots and ELISA assays and which interferes with the binding of the peptide to its target.

Preferred in the context of the present invention is a monoclonal antibody that selectively binds to the antimicrobial peptide of the present invention, more particularly and preferably selectively to an antimicrobial peptide according to any of the sequence according to SEQ ID No. 1 to SEQ ID No. 8 and/or antimicrobially active parts thereof. Most preferred is a monoclonal antibody which immunologically recognizes all of the sequences according to SEQ ID No. 1 to SEQ ID No. 8 and/or antimicrobially active parts thereof. Further preferred is an antibody that binds competitively with the peptide according to the invention to its target on the microorganism, in those cases such an antibody or fragment thereof could be used as a drug.

A “fragment” of a ligand, in particular a fragment of an antibody, shall mean a moiety that is derived from the ligand that is still capable of binding to the respective cellular marker (for example, the antimicrobial peptide). Particular examples for antibodies are scFV-fragments and other antibody-derived peptides that can bind to the respective marker. In a preferred embodiment, the binding of the fragment leads to the same biological effect(s) as the binding of the full-length (or sized) ligand, preferably the binding of the peptide according to the invention. The use of anti-idiotypic antibodies is also contemplated to be in the scope of the present invention.

Modifications and changes may be made in the structure of the peptides of the present invention and DNA segments which encode them and still obtain a functional molecule that encodes a protein or peptide with desirable characteristics. The amino acids changes may be achieved by changing the codons of the DNA sequence. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

In addition, amino acid substitutions are also possible without affecting the antimicrobial effect of the isolated peptides of the invention, provided that the substitutions provide amino acids having sufficiently similar properties to the ones in the original sequences.

Accordingly, acceptable amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine. The isolated peptides of the present invention can be prepared in a number of suitable ways known in the art including typical chemical synthesis processes to prepare a sequence of polypeptides.

Another aspect of the present invention then relates to a nucleic acid having a nucleotide sequence that encodes for the antimicrobial peptide according to the present invention, and in particular for a peptide having the amino acid sequence as set forth in any one of SEQ ID NO: 1 to 8 and/or antimicrobially active parts thereof, or a complementary nucleotide sequence thereof. The nucleic acid molecules of the invention can be DNA, cDNA, PNA, CNA, RNA, cDNA, genomic DNA, synthetic DNA, or combinations thereof, and can be double-stranded or single-stranded, the sense and/or an antisense strand. Segments of these molecules are also considered within the scope of the invention, and can be produced by, for example, the polymerase chain reaction (PCR) or generated by treatment with one or more restriction endonucleases. A ribonucleic acid (RNA) molecule can be produced by in vitro transcription.

The nucleic acid molecules of the invention can contain naturally occurring sequences, or sequences that differ from those that occur naturally, but, due to the degeneracy of the genetic code, encode the same peptide (for example, the peptides with SEQ ID NOs: 1 to 8 and/or antimicrobially parts thereof). In addition, these nucleic acid molecules are not limited to coding sequences, e.g., they can include some or all of the non-coding sequences that lie upstream or downstream from a coding sequence.

The nucleic acid molecules of the invention can be synthesized in vitro (for example, by phosphoramidite-based synthesis) or obtained from a cell, such as the cell of a bacterium or mammal. The nucleic acids can be those of a human but also derived from a non-human primate, mouse, rat, guinea pig, cow, sheep, horse, pig, rabbit, dog, or cat as long as they fulfill the criteria set out above. Combinations or modifications of the nucleotides within these types of nucleic acids are also encompassed.

In addition, the isolated nucleic acid molecules of the invention encompass segments that are not found as such in the natural state. Thus, the invention encompasses recombinant nucleic acid molecules incorporated into a vector (for example, a plasmid or viral vector) or into the genome of a heterologous cell (or the genome of a homologous cell, at a position other than the natural chromosomal location). Recombinant nucleic acid molecules and uses therefore are discussed further below.

A nucleic acid belonging to an antimicrobial peptide as disclosed herein or a protein can be identified based on its similarity to the relevant antimicrobial peptide gene or protein, respectively. For example, the identification can be based on sequence identity. In certain preferred embodiments the invention features isolated nucleic acid molecules which are at least 50% (or 55%, 65%, 75%, 85°/a, 95%, or 98%) identical to: (a) a nucleic acid molecule that encodes the peptide of SEQ ID NOs: 1 to 8 and/or antimicrobially parts thereof, and (b) a nucleic acid molecule which includes a segment of at least 30 (e.g., at least 30, 40, 50, 60, 80, 100, or 125 nucleotides of a nucleic acid molecule that encodes the peptide of SEQ ID NOs: 1 to 8 and/or antimicrobially parts thereof.

The determination of percent identity between two sequences is accomplished using the mathematical algorithm of Karlin and Altschul, Proc. Natl. Acad. Sci. USA 90, 5873-5877, 1993. Such an algorithm is incorporated into the BLASTN and BLASTP programs of Altschul et al. (1990) J. Mol. Biol. 215, 403-410. BLAST nucleotide searches are performed with the BLASTN program, score=100, wordlength=12, to obtain nucleotide sequences homologous to antimicrobial peptide-encoding nucleic acids. BLAST protein searches are performed with the BLASTP program, score=50, wordlength=3, to obtain amino acid sequences homologous to the antimicrobial peptide. To obtain gapped alignments for comparative purposes, Gapped BLAST is utilized as described in Altschul et al. (1997) Nucleic Acids Res. 25, 3389-3402. When utilizing BLAST and Gapped BLAST programs, the default parameters of the respective programs.

Hybridization can also be used as a measure of homology between two nucleic acid sequences. A nucleic acid sequence encoding any of the antimicrobial peptides disclosed herein, or a portion thereof, can be used as a hybridization probe according to standard hybridization techniques. The hybridization of an antimicrobial peptide probe to DNA or RNA from a test source (e.g., a mammalian cell) is an indication of the presence of the relevant peptide DNA or RNA in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6× sodium chloride/sodium citrate (SSC) at 45° C., followed by a wash in 0.2×SSC, 0.1% SDS at 65° C.

The DNA (or in the case of retroviral vectors, RNA) is then expressed in a suitable host to produce a polypeptide comprising the compound of the invention. Thus, the DNA encoding the polypeptide constituting the compound of the invention may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. Nos. 4,440,859 issued 3 Apr. 1984 to Rutter et al, 4,530,901 issued 23 Jul. 1985 to Weissman, 4,582,800 issued 15 Apr. 1986 to Crowl, 4,677,063 issued 30 Jun. 1987 to Mark et al, 4,678,751 issued 7 Jul. 1987 to Goeddel, 4,704,362 issued 3 Nov. 1987 to Itakura et al, 4,710,463 issued 1 Dec. 1987 to Murray, 4,757,006 issued 12 Jul. 1988 to Toole, Jr. et al, 4,766,075 issued 23 Aug. 1988 to Goeddel et al and 4,810,648 issued 7 Mar. 1989 to Stalker.

The DNA (or in the case of retroviral vectors, RNA) encoding the polypeptide constituting the compound of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

The present invention also relates to a host cell transformed with a polynucleotide vector construct of the present invention. The host cell can be either prokaryotic or eukaryotic. Bacterial cells may be preferred prokaryotic host cells in some circumstances and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast, insect and mammalian cells, preferably vertebrate cells such as those from a mouse, rat, monkey or human fibroblastic and kidney cell lines. Yeast host cells include YPH499, YPH500 and YPH501 which are generally available from Stratagene Cloning Systems, La Jolla, Calif. 92037, USA. Preferred mammalian host cells include Chinese hamster ovary (CHO) cells available from the ATCC as CCL61, NIH Swiss mouse embryo cells NIH/3T3 available from the ATCC as CRL 1658, monkey kidney-derived COS-1 cells available from the ATCC as CRL 1650 and 293 cells which are human embryonic kidney cells. Preferred insect cells are Sf9 cells which can be transfected with baculovirus expression vectors.

Transformation of appropriate cell hosts with a DNA construct of the present invention is accomplished by well known methods that typically depend on the type of vector used. With regard to transformation of prokaryotic host cells, see, for example, Cohen et al (1972) Proc. Natl. Acad. Sci. USA 69, 2110 and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA. Electroporation is also useful for transforming and/or transfecting cells and is well known in the art for transforming yeast cell, bacterial cells, insect cells and vertebrate cells.

Successfully transformed cells, i.e. cells that contain a DNA construct of the present invention, can be identified by well known techniques. For example, cells resulting from the introduction of an expression construct of the present invention can be grown to produce the polypeptide of the invention. Cells can be harvested and lysed and their DNA content examined for the presence of the DNA using a method such as that described by Southern (1975) J. Mol. Biol. 98, 503 or Berent et al (1985) Biotech. 3, 208. Alternatively, the presence of the protein in the supernatant can be detected using antibodies as described below.

Many expression systems are known, including bacteria (for example E. coli and Bacillus subtilis), yeasts (for example Saccharomyces cerevisiae), filamentous fungi (for example Aspergillus), plant cells, animal cells and insect cells, as above.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention. Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

In another aspect of the present invention, the present invention provides a competitive screening assay method for screening for competitive ligands of the target of the antimicrobial peptide according to the present invention, comprising the steps of: a) incubating said peptide with a putative competitive ligand and said microbe, b) measuring the binding between said peptide and said microbe in the presence and absence of said competitive ligand, and c) identifying said ligand based on the binding as measured in step b). Preferred is such a screening assay, wherein furthermore an antimicrobial activity of said ligand as identified is measured. Further preferred is an assay, wherein said peptide comprises a detectable label, such as a dye, enzyme, radioactive or fluorogenic marker. Furthermore, peptide-nucleic acid fusions are possible.

Even further preferred is an assay, wherein said microbe is selected from a fungus, in particular a yeast, in particular C. albicans or a bacterium, such as a gram-negative E. coli, a gram-positive Staphylococcus aureus, Corynebacterium sp., Microsporum spp., Epidermophyton spp., Cryptococcus neoformans, Trichophyton spp., Sporothrix schenkii and Aspergillus fumigatus.

In one embodiment, these ligands can be identified based on the structural similarities of the antimicrobial peptide with other proteins. Examples for the generation of such ligands are described in the literature (as mentioned also herein) and are well known to the person of skill.

During the course of the assay of the method according to the invention, the ligand will initially bind or attach to the site of binding of the antimicrobial peptide of the invention. The ligand can either bind directly or indirectly to the site, i.e. via cofactors that can be present, such as certain ions or protein factors that promote the attachment of the ligand to the site, and therefore support the function of the ligand. “Binding” can occur via a covalent or non-covalent attachment of the ligand or group of ligands to the binding site. Based on the binding properties of the screened ligands, a first pre-selection of ligands can be performed, in which a non-binding ligand is screened in a second “round” of screening using a set of co-factors. If still no binding occurs, the ligand will be classified as “non-binding” and disregarded in further screenings. Such pre-selection will be encompassed by the terms “screening”, “measuring” and/or “determining” in the general context of this invention. A ligand that shows an in-vitro action should in vivo preferably not further interact with components of the patients' or test (model) organisms' body, e.g. within the bloodstream, lung and/or heart of the patient or test organism.

In general, assays to determine a binding and biological effect of a ligand to a specific target (in this case the antimicrobial effect) are well known to the person skilled in the art and can be found, for example, in U.S. Pat. Nos. 4,980,281, 5,266,464 and 5,688,655 to Housey for phenotypic changes of cells after incubation with a screening ligand. Furthermore, U.S. Pat. No. 5,925,333 to Krieger at al. describes methods for modulation of lipid uptake and related screening methods.

The method of screening according to the present invention can be performed in several different formats. One embodiment is a method, wherein the assay is performed in vitro. The screening assays of the present invention preferably involve the use of host cell-lines (as described above) and other cells, as long as these cells express the target of the antimicrobial peptide according to the invention. How to produce such recombinant cells is well known to the skilled artisan and is further described above and in the respective literature.

An additional embodiment of the present invention relates to a method wherein the assay is performed in vivo. Preferably, the assay is performed in a mouse or rat. In general, the in vivo assay will not be substantially different from the above-mentioned in vitro assay. In a general screening assay for ligands of the site of the antimicrobial peptide will be provided in that the ligand to be tested is/are administered to a mouse or a rat. Then, it will be determined, if said ligand leads to an inhibition of the binding of the antimicrobial peptide, compared to the absence of the ligand to be tested, wherein a difference identifies a ligand that is competitive for the binding activity of the antimicrobial peptide. Of course, these assays can be performed in other non-human mammals as well. In a second step, the antimicrobial activity of the competitive ligand itself can be tested, e.g. in an assay as described herein below.

An additional embodiment of the present invention relates to a method according to the invention, wherein said ligand is selected from a library of naturally occurring or synthetic compounds which are randomly tested for competitive binding to the binding site on the microbe. Such libraries and the methods how to build up such a library, as well as methods for using these libraries for the screening of candidate ligands are well known to the person skilled in the art and further described in the respective literature. Furthermore, some of these libraries are commercially available. The present invention contemplates high throughput screening of competitive ligands for the site. The ligands as described above, and modifications of said ligands, including analogues, derivatives, fragments, active moieties, and the like, may be screened using methods and systems of the present invention.

Potential antagonists include small organic molecules, peptides, polypeptides and antibodies. For example, a compound that inhibits the antimicrobial peptide binding activity may be a small molecule that binds to and occupies the binding site of the antimicrobial peptide, thereby preventing binding to cellular binding molecules, to prevent normal biological activity. Examples of small molecules include, but are not limited to, small organic molecule, peptides or peptide-like molecules. Other potential antagonists include antisense molecules. Preferred antagonists include compounds related to and variants or derivatives of the antimicrobial peptides or portions thereof. The nucleic acid molecules described herein may also be used to screen compounds for antimicrobial activity.

Another preferred embodiment of the present invention relates to a method for the production of a pharmaceutical formulation, comprising the steps of: a) performing a screening method as above, and b) formulating the ligand as identified with a pharmaceutically acceptable carrier and/or excipient. Such formulations therefore include, in addition to the ligand/antibody, a physiologically acceptable carrier or diluent, possibly in admixture with one or more other agents such as other antibodies or drugs, such as an antibiotic. Suitable carriers include, but are not limited to, physiological saline, phosphate buffered saline, phosphate buffered saline glucose and buffered saline. Alternatively the ligand, e.g. the antibody, may be lyophilized (freeze dried) and reconstituted for use when needed by the addition of an aqueous buffered solution as described above. Routes of administration are routinely parenteral, including intravenous, intramuscular, subcutaneous and intraperitoneal injection or delivery. The administration can be systemic and/or locally.

Preferred pharmaceutical compositions according to the present invention comprise at least one peptide according to the present invention, an antibody according to the present invention, a nucleic acid according to the present invention or an expression vector according to the present invention, and a pharmaceutically acceptable carrier as above.

The pharmaceutical composition is used for topical or parenteral administration, such as subcutaneous, intradermal, intraperitoneal, intravenous, intramuscular or oral administration. For this, the peptides are dissolved or suspended in a pharmaceutically acceptable, preferably aqueous carrier. In addition, the composition can contain excipients, such as buffers, binding agents, blasting agents, diluents, flavors, lubricants, etc. The composition can be used for a prevention, prophylaxis and/or therapy of antimicrobial, and in particular fungal diseases.

The pharmaceutical preparation of the present invention, containing at least one of the peptides of the present invention, a nucleic acid according to the invention, an antibody of the present invention, or an expression vector according to the invention, is administered to a patient that suffers from an antimicrobial, and in particular fungal disease, in particular C. albicans infection. Preferably, said microbial infection is selected from cystic fibrosis lung infection; joint sepsis, ocular infections, periodontal disease, STDs, otitis externa, cutaneous infections, burn infections, vaginal infections, diabetic foot ulcers, and a fungal infection, in particular a candidosis.

In general, the peptides that are present in the pharmaceutical composition according to the invention have the same properties as described above for peptides of the present invention. Thus, they can have an overall length of between 8 and 100, preferably between 8 and 30, and most preferred between 8 and 12 amino acids. Furthermore, at least one peptide according to the invention can include non-peptide bonds. Furthermore, the respective nucleic acids can encode for between 8 and 100, preferably between 8 and 30, and most preferred between 8 and 12 amino acids. Most preferred is a pharmaceutical composition according to the invention that comprises a peptide consisting of amino acid sequences according to SEQ ID No. 1 or 2 or an antimicrobial peptide derived from SEQ ID No. 3 to 8.

The dosage of the peptide or ligand according to the present invention to be administered to a patient suffering from the present diseases will vary with the precise nature of the condition being treated and the recipient of the treatment. The dose will generally be in the range of about 0.1 to about 100 mg for an adult patient, usually administered daily for a period between 1 and 30 days. The preferred daily dose is 1 to 10 mg per day, although in some instances larger doses of up to 40 mg per day may be used. Preferably the dosage will be applied in such a manner that the ligand is present in the medicament in concentrations that provide in vivo concentrations of said ligand in a patient to be treated of between 0.01 mg/kg/day and 1 mg/kg/day. Most preferred is a pharmaceutical composition, wherein the peptide or ligand according to the invention is present in an amount to achieve a concentration in vivo of 12 μg/ml or above. The pharmaceutical preparation of the present invention can further contain at least one host defense molecule, such as lysozyme and/or lactoferrin.

Most preferred is a pharmaceutical composition according to the invention in the form of an ointment, gel or skin crème for topical treatment of the microbial infection. When applied topically, the peptide compositions according to the present invention may be combined with other ingredients, such as carriers and/or adjuvants. The peptides according to the present invention may also be covalently attached to a protein carrier, such as albumin, or to a prosthetic implant so as to minimize diffusion of the peptides. The nature of such other ingredients can be readily determined by the skilled artisan and only requires that they must be pharmaceutically acceptable, efficacious for the intended administration and cannot degrade the active ingredients of the compositions according to the present invention. When the peptide compositions of this invention are applied to a site of topical infection, they may act as an irritant (which would stimulate influx of scavenger cells). The peptide compositions can also be in the form of ointments or suspensions, preferably in combination with purified collagen. The peptide compositions according to the present invention also may be impregnated into transdermal patches, plasters and bandages, preferably in a liquid or semi-liquid form.

Another aspect of the present invention relates to the peptide according to the present invention, the antibody according to the present invention, the nucleic acid according to the present invention or the expression vector according to the present invention or the pharmaceutical composition according to the present invention for use in medicine. Preferred is a use of the pharmaceutical composition according to the present invention as an anti-microbial agent.

Another aspect of the present invention relates to the use of a peptide according to the present invention, the antibody according to the present invention, the nucleic acid according to the present invention or the expression vector according to the present invention or the pharmaceutical composition according to the present invention for the manufacture of a medicament (drug?) for the prevention and/or treatment of microbial infections, preferably fungal infections, in particular a candidal infection.

The present invention provides a method for the prevention and/or treatment of a human subject suffering from an antimicrobial infection and/or fungal infection and/or C. albicans infection, which comprises administering to the said subject an effective amount of an agent selected from a peptide according to the invention, an antibody according to the invention, a nucleic acid according to the invention, an expression vector according to the invention, or the pharmaceutical composition according to the invention. Suitable formulations, routes of administrations and dosages are indicated above and are further laid out in the following examples. Most preferred is the treatment as an anti-C. albicans medication.

The present invention further provides a diagnostic kit for diagnosing a microbe, comprising a peptide according to the invention, optionally together with suitable labels and dyes. comprising a peptide according to the present invention, an antibody according to the present invention, and/or a nucleic acid according to the present invention, optionally together with suitable labels and dyes. Thus, the binding of the peptide or the above-described competitive ligand according to the invention can be used as a diagnostic agent for diagnosing a microbe, such as a fungus, in particular a yeast, in particular C. albicans.

As indicated above, GAL has been shown to have a widespread distribution in the central and peripheral nervous systems of many mammalian species (Berger A, Kofler B, Santic R, Zipperer E, Sperl W and Hauser-Kronberger C: 1251-labeled galanin binding sites in congenital innervation defects of the distal colon. Acta Neuropathol (Berl) 105:43-48, 2003a; Berger A, Santic R, Almer D, et al: Galanin and galanin receptors in human gliomas. Acta Neuropathol (Berl) 105:555-560, 2003b; Jacobowitz D M, Kresse A and Skofitsch G: Galanin in the brain: chemoarchitectonics and brain cartography—a historical review. Peptides 25:433-464, 2004). Consistent with its widespread distribution, physiological studies in a number of animal model systems and in man have allowed identification of a diversity of biological effects. These include effects on the secretion of hormones such as insulin, glucagon, and growth hormone, effects on neurotransmitter release in the hippocampus, inhibition of memory and learning, central and peripheral effects on the cardiovascular system, stimulation of appetite and analgesic effects in response to nerve injury (Mazarati A M: Galanin and galanin receptors in epilepsy. Neuropeptides 38:331-343, 2004; Vrontakis M E: Galanin: a biologically active peptide. Curr Drug Targets CNS Neurol Disord 1:531-541, 2002).

Ji et. al found galanin-like immunoreactivity (GAL-LI) and GAL mRNA in the dermis and epithelium of the rat hind paw especially during inflammation (Ji R R, Zhang X, Zhang Q, Dagerlind A, Nilsson S, Wiesenfeld-Hallin Z and Hokfelt T: Central and peripheral expression of galanin in response to inflammation. Neuroscience 68563-576, 1995). In the human skin GAL was detected by the inventor mainly in the epidermis and in sweat glands in a non-neuronal distribution (Kofler B, Berger A, Santic R, et al: Expression of neuropeptide galanin and galanin receptors in human ski. J Invest Dermatol 122:1050-1053, 2004a). To a lesser extent it was found in nerve fibre bundles and smooth muscles. Real Time-RT-PCR and northern blot analysis of human primary cultured keratinocytes revealed detectable levels of GAL mRNA. In vitro receptor autoradiography using [¹²⁵I]-labelled GAL detected binding sites in the dermis, mainly around sweat glands but also on muscle tissue, in nerve fibre bundles and in close association to blood vessels, but not in the membranes of cultured human dermal micro vascular endothelial cells, fibroblasts and keratinocytes. The study established GAL as a major neuropeptide in human skin (Kofler B, Berger A, Santic R, et al: Expression of neuropeptide galanin and galanin receptors in human ski. J Invest Dermatol 122:1050-1053, 2004a).

To the knowledge of the inventors, to date no local function for GAL produced in the epidermis has been described. Since, other (neuro)peptides have been shown to possess an antimicrobial activity in the skin, the inventors started to test the possibility that fragments of ppGAL have antimicrobial activity against bacterial and fungal pathogens.

Indications for a potential antimicrobial function of ppGAL are the following characteristics, which are common features of other AMPs (Bals R: Epithelial antimicrobial peptides in host defense against infection. Respir Res 1: 141-150, 2000):
high level of expression in epidermis (Kofler B, Berger A, Santic R, et al: Expression of neuropeptide galanin and galanin receptors in human ski. J Invest Dermatol 122:1050-1053, 2004a), which for example parallels the expression of the antimicrobial neuropeptide α-MSH
sites of potential microbial entry in the skin, such as follicular structures and sweat glands express GAL (Kofler B, Berger A, Santic R, et al: Expression of neuropeptide galanin and galanin receptors in human ski. J Invest Dermatol 122:1050-1053, 2004a)
up-regulation of ppGAL mRNA by pro-inflammatory cytokines; (Kofler B, Berger A, Santic R, et al: Expression of neuropeptide galanin and galanin receptors in human ski. J Invest Dermatol 122:1050-1053, 2004a)
arginin/proline rich region in ppGAL (64-89) (Evans H F and Shine J: Human galanin: molecular cloning reveals a unique structure. Endocrinology 129:1682-1684, 1991)
posttranslational processing of the precursor molecule is required for a functional peptide
lack of GAL receptor expression in vicinity of epidermal layer and on epidermal cells (Kofler B, Berger A, Santic R, et al: Expression of neuropeptide galanin and galanin receptors in human ski. J Invest Dermatol 122:1050-1053, 2004a) indicates a non-neuro (endocrine) function
   Candida albicans and its Pathogenesis

Fungi are eukaryotic organisms with approximately 300 000 different species. Of these, about 200 are potential parasites, with only a few of these affecting humans. Fungal diseases of mammals, mycoses, range from the common mild cutaneous or subcutaneous skin infections, such as athletes foot, to the potentially lethal acute or chronic infection of deep tissues that are typically caused by Candida species. Of the Candida species afflicting humans, Candida albicans is by far the most common. Candida albicans belongs to the class Ascomycetes and the family, Saccharomycetaceae. This yeast can live as harmless commensal in many different body locations, and is carried in almost half of the population. However, in response to a change in the host environment, C. albicans can convert from a benign commensal into a disease-causing pathogen, causing infections in the oral, gastrointestinal and genital tracts. The infection caused by C. albicans can be defined in two broad categories, superficial mucocutaneous and systematic invasive, which involves the spread of C. albicans to the blood stream (candidemia) and to the major organs. Systemic candidemia is often fatal. Superficial infections affect the various mucous membrane surfaces of the body such as in oral and vaginal thrush. The incidence of vulvovaginal candidiasis (thrush) has increased approximately 2-fold in the last decade. Approximately 75% of all women experience a clinically significant episode of vulvovaginal candidiasis (VVC) at least once during the reproductive period.

Numerous virulence factors have been attributed to the pathogenicity of C. albicans. These include dimorphism, phenotypic switching and immune interference.

### Dimorphism and Phenotypic Switching:

Candida albicans is a diploid asexual and dimorphic fungus and depending upon environmental conditions, can exist as unicellular yeast (blastospores and chlamydospores) as well as in different filamentous forms (hypha, pseudo-hyphae). Several studies suggest that the ability of C. albicans to switch between the yeast and mycelial forms is an important virulence factor. Increased adherence to oropharyngeal surfaces has been observed for the mycelial form. Decreased adherence has been demonstrated by a non-germ tube producing variant in experimental vaginitis.

As already mentioned the GMAP inhibits the pathogenic phenotypic switch of C. albicans most likely by interference with the signalling pathway inducing this switch. This function does not dramatically interfere with the growth of the pathogen but inhibits its switch to a disease causing fungus. Therefore, the pressure to overcome this lack is not high for the fungus since its growth per se is not affected. Most other known antifungal agents are killing the fungus and therefore gain of resistance is important for the pathogen and is observed especially in hospital acquired infections.

Furthermore, the peptides of the present invention may be useful to inhibit microbial colonization. For example, the peptides may be delivered and expressed by eukaryotic cells in vivo, via transfection using viral vectors. The continued expression of the peptides in the cells and secretion into their environment interferes with colonization of microbes and prevent microbial infection. Cells expressing the peptides according to the present invention may be able to continuously combat the colonization of a range of pathogenic microbes.

In the immunocompromised host (neonate, chemotherapy, HIV) prevention of a potential invasive fungal infection can be carried out by prophylactical application (upregulation) of GMAP on epithelial surfaces.

Side effects of GMAP treatment are not many to be expected, since it is an endogenous peptide and does not have any major other functions in the body. Therefore, compared to other neuropeptides with antimicrobial activity which also are able to function via different receptors, for GMAP no neuro/endocrine side effects are expected.

The peptides according to the present invention are not fungicidal, but inhibit its conversion to a pathogenic organism. Thus, resident candida populations on outer and inner body surfaces are not harmed. This is important, because the comensural Candida population is integral part of the microbial population of the body surfaces. Specific killing C. albicans can alter the physiological cross talk between fungi and bacteria. This is even more of importance as fungicidal drugs have to be used up to one year to treat candidal infections of the nails or to prevent candidosis in patients with a CD4 T cell count below 100.

Furthermore, the known problems of growing resistance to known small molecule drugs like amphotericin B, 5-flucytosine, fluconazol, itraconazol can be circumvented by using the peptide.

In addition, the sometimes fatal side effects of the known drugs including allergic exanthemas, hematopoetic alterations, gastrointestinal discomfort and hepatic toxicity are not to be expected with the peptide and its formulations. Since the peptide according to the present invention will not be degraded via the cytochrom p450 pathway in the liver interference with other medications of a given patient (e.g. anticoagulants, antidepressive medication) will not be encountered.

It should be understood that the features of the invention as disclosed and described herein can be used not only in the respective combination as indicated but also in a singular fashion without departing from the intended scope of the present invention.

The invention will now be described in more detail by reference to the following Figures, the Sequence listing, and the Examples. The following examples are provided for illustrative purposes only and are not intended to limit the invention.

SEQ ID No 1 to SEQ ID No 2 show preferred peptide sequences of the antimicrobial peptides according to the present invention.

SEQ ID No 3 to SEQ ID No 8 show GMAP sequences from which additional antimicrobial peptides according to the present invention can be derived.

FIG. 1 shows the structure of the pre-pro galanin (ppGAL) mRNA. P: pro-peptide; Galanin: mature galanin peptide; GMAP: galanin-message associated peptide. Dibasic proteolytic cleavage sites are indicated by arrows. Black bars indicate peptide fragments used in antimicrobial assays (see below).

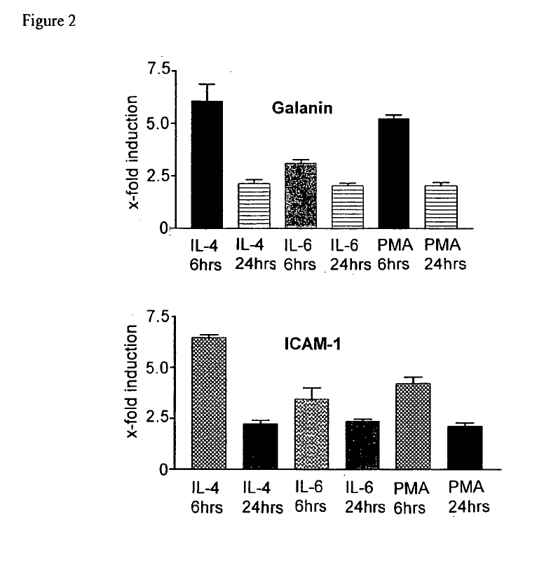

FIG. 2 shows up-regulation of ppGAL mRNA by pro-inflammatory cytokines (A) in comparison with the adhesion molecule ICAM-1 as positive control in primary cultured human keratinocytes.

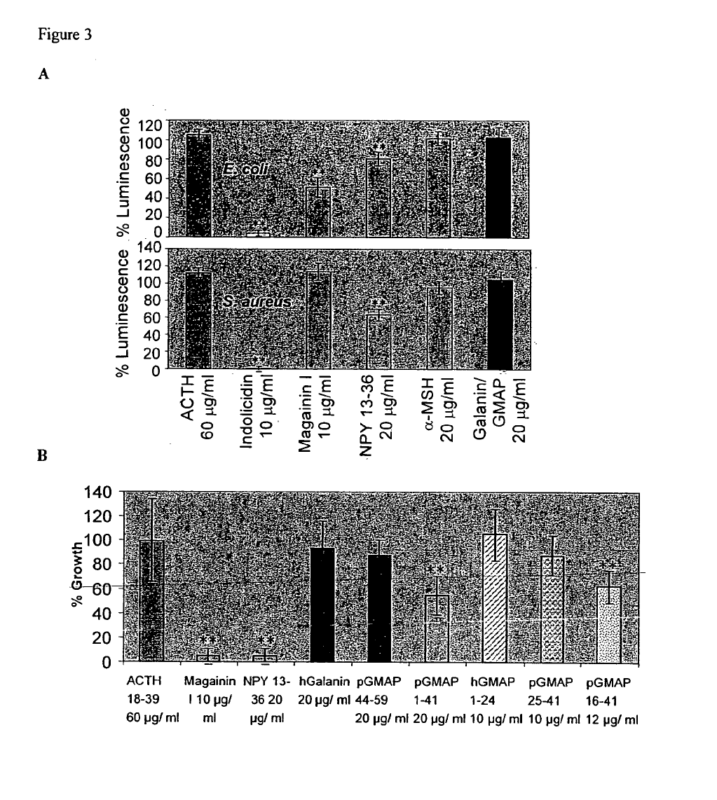

FIG. 3 shows that none of the peptide fragments tested of the ppGal gene did reveal antibacterial activity (A). Against C. albicans the mix of pp-Galanin-fragments (1-41; 16-41) displayed a significant inhibitory growth effect (C). Significant changes in growth are indicated by ** p<0.05.

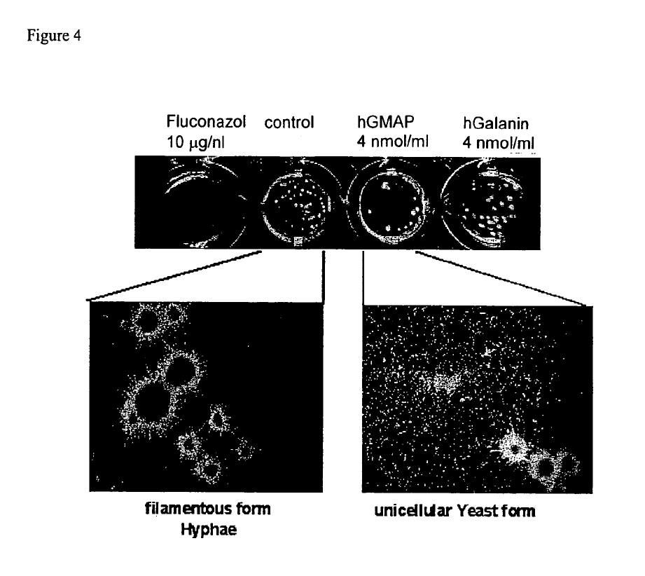

FIG. 4 shows cultures of Candida albicans were treated for 48 hrs either with the common antifungal substance fluconazol, GMAP 16-42 or Galanin. Microscopic examination of the cultures revealed that hyphae were only visible in the untreated and galanin treated wells. In the GMAP treated wells mainly a dense suspension of unicellular yeast forms were observed.

### EXAMPLES

### Antimicrobial Activity of ppGAL

The inventors analyzed the antimicrobial activity of peptide fragments corresponding to ppGAL against gram-negative E. coli gram-positive Staphylococcus aureus and Corynebacterium sp. and the yeast Candida albicans and Aspergillus fumigatus. Positive controls were the antimicrobial peptides Indolicidin, NPY 13-36 and Magainin I, as a negative control served the Adrenocorticotropic hormone (ACTH) 18-39 (Cutuli M, Cristiani S, Lipton J M and Catania A: Antimicrobial effects of alpha-MSH peptides. J Leukoc Biol 67:233-239, 2000; Selsted M E, Novotny M J, Morris W L, Tang Y Q, Smith W and Cullor J S: Indolicidin, a novel bactericidal tridecapeptide amide from neutrophils. J Biol Chem. 267:4292-4295., 1992; Shimizu M, Shigeri Y, Tatsu Y, Yoshikawa S and Yumoto N: Enhancement of antimicrobial activity of neuropeptide Y by N-terminal truncation. Antimicrob Agents Chemother 42:2745-2746, 1998; Zasloff M: Magainins, a class of antimicrobial peptides from Xenopus skin: isolation, characterization of two active forms, and partial cDNA sequence of a precursor. Proc Natl Acad Sci USA 84:5449-5453, 1987).

None of the peptide fragments tested of the ppGal gene did reveal antibacterial activity. Against C. albicans the mix of pp-Galanin-fragments displayed a significant inhibitory growth effect. Further experiments identified GMAP 1-41 as the active compound. The inventors were able to narrow the antimicrobial core sequence down to GMAP 16-41. The antifungal activity can be found already at a concentration of 12 pg/ml (4 μM).

Cultures of Candida albicans were treated for 24 and 48 hrs either with the common antifungal substance fluconazol, GMAP 16-42 or Galanin. Microscopic examination of the cultures revealed that hyphae were only visible in the untreated and galanin treated wells. In the GMAP treated wells only a dense suspension of yeast forms were observed. Without wanting to be bound by theory, for Candida these findings indicate that GMAP appears not to significantly affect the proliferation of Candida per se, but inhibits the switch to pathogenic hyphal forms.

These studies establish ppGAL as a new component of the innate immune system which has implications for therapeutic strategies. Either directly by applying a GMAP analogue or indirectly by up-regulation of the expression of GMAP levels to inhibit pathogenic dissemination of the fungus.

This mechanism of action is different to common antifungal substances like fluconazol, which are cytotoxic to the pathogen by inhibiting growth or direct killing. Inhibition of the transition to pathogenic forms in combination with common cytotoxic drugs like fluconazol might lower the risk of pathogen dissemination. Furthermore, rising problems with resistance to presently used antifungal drugs might be solved by using drugs with a different pathway of action like we present here for GMAP.

Preferred aspects of the invention thus are selected from the use of GMAP and fragments as antifungal substances especially against Candida species but also other fungal pathogens with features of dimorphism and phenotypic switching, the use of GMAP and fragments to inhibit switch and growth of hyphal forms of Candida, and the use of any substances leading to an up-regulation of GMAP gene expression in epithelial and endothelial cells resulting in a decrease of fungal virulence.

1. An antimicrobial peptide, comprising the amino acid sequence of human galanin message associated peptide (GMAP) amino acids 16-41 (SEQ ID No. 1), or a homolog, ortholog, chemically modified variant or antimicrobially active variant thereof, wherein the peptide is not complete GMAP.

2. The antimicrobial peptide according to claim 1, comprising the amino acid sequence of human galanin message associated peptide (GMAP) amino acids 1-41 (SEQ ID No. 2), or a homolog, ortholog, chemically modified variant or antimicrobially active variant thereof, wherein the peptide is not complete GMAP.

3. The antimicrobial peptide according to claim 1, wherein said peptide consists or consists essentially of the amino acid sequence 16-41 according to any of SEQ ID No. 3 to SEQ ID No. 8 or a chemically modified variant thereof.

4. The antimicrobial peptide according to claim 1, wherein said peptide has a length of between 8 and 100 amino acids.

5. The antimicrobial peptide according to claim 1, wherein said peptide includes non-peptide bonds.

6. The antimicrobial peptide according to claim 1, wherein said peptide is a fusion protein.

7. An antibody or fragment thereof that is immunologically reactive with the antimicrobial peptide according to claim 1.

8. A nucleic acid, encoding a peptide according to claim 1.

9. The nucleic acid according to claim 8 which is DNA, cDNA, PNA, CNA, RNA or a combination thereof.

10. An expression vector capable of expressing a nucleic acid according to claim 8.

11. A host cell comprising a nucleic acid according to claim 8.

12. A method for screening for compounds that competitively bind to a microbe with the antimicrobial peptide according to claim 1 comprising the steps of: a) incubating said peptide with a putative competitive ligand and said microbe, b) measuring the binding between said peptide and said microbe in the presence and absence of said competitive ligand, and c) identifying said ligand based on the binding as measured in step b).

13. The method according to claim 12, wherein furthermore an antimicrobial activity of said ligand is measured.

14. The method according to claim 12, wherein said peptide comprises a detectable label.

15. The method according to claim 12, wherein said microbe is selected from C. albicans, a gram-negative E. coli, a gram-positive Staphylococcus aureus, Corynebacterium sp., Microsporum spp., Epidermophyton spp., Cryptococcus neoformans, Trichophyton spp., Sporothrix schenkii and Aspergillus fumigatus.

16. A method for the production of a pharmaceutical formulation, comprising the steps of: a) performing a method according to claim 12, and b) formulating said ligand as identified with a pharmaceutically acceptable carrier and/or excipient.

17. A pharmaceutical composition comprising at least one peptide according to claim 1, an antibody or fragment thereof that is immunologically reactive with said peptide or a nucleic acid, encoding said peptide and a pharmaceutically acceptable carrier.

18. The pharmaceutical composition according to claim 17, further comprising at least one host defense molecule.

19. The pharmaceutical composition according to claim 17, wherein the peptide is present in an amount to achieve a concentration in vivo of 12 μg/ml.

20. The pharmaceutical composition according to claim 17, in the form of an ointment, gel or skin crème.

21. (canceled)

22. A method for preventing and/or treating a microbial infection wherein said method comprises administering to a subject in need of such prevention and/or treatment a pharmaceutical composition according to claim 17 as an anti-microbial agent.

23. Use of a peptide according to claim 1, an antibody or fragment thereof that is immunologically reactive with said peptide or a nucleic acid encoding said peptide for the manufacture of a medicament for the prevention and/or treatment of microbial infections.

24. The method according to claim 22, wherein said microbial infection is selected from cystic fibrosis lung infection; joint sepsis, ocular infections, periodontal disease, STDs, otitis externa, cutaneous infections, burn infections, vaginal infections, diabetic foot ulcers, and fungal infections.

25. A diagnostic kit for diagnosing a microbe, comprising a peptide according to claim 1, an antibody or fragment thereof that is immunologically reactive with the antimicrobial peptide according to claim 1, and/or anucleic acid, encoding a peptide according to claim 1, optionally together with suitable labels and dyes.

26. The kit according to claim 25, wherein said microbe is a fungus, in particular a yeast, in particular C. albicans.
